(19) United States
(12) Patent Application Publication
(10) Pub. No.: US 2002/0100474 A1
(43) Pub. Date: Aug. 1, 2002

(54) Respiratory flow sensor

(76) Inventors: Bernd Kellner, Lubeck (DE); Martin Meyer, Lubeck (DE); Jochen Daske, Lubeck (DE); Ralf Dittmann, Lubeck (DE)

Correspondence Address: McGLEW AND TUTTLE, P.C., SCARBOROUGH STATION, SCARBOROUGH, NY, 10510-0827, US

(21) Appl. No.: 10/039,032

(22) Filed: Jan. 2, 2002

(30) Foreign Application Priority Data
Feb. 1, 2001 (DE) 101 04 462.3

(51) Int. Cl. A62B 7/00; A62B 9/00; A61M 15/00; A61M 16/00; A62B 18/00; A61B 5/08

(52) U.S. Cl. 128/200.24; 600/538

(57) ABSTRACT

An improved respiratory flow sensor with a tubular housing (1) in the form of a venturi tube and with a measuring insert (5) that can be inserted into same with at least one hot wire (2, 3). The tube and insert are components of long durability which can be autoclaved and consequently reused. The measuring insert (5) is replaceable fastened in the housing (1) by means of a snap connection and is formed of a high-temperature plastic that can be autoclaved by use of hot steam.

### FIELD OF THE INVENTION

The present invention pertains respirators and anesthesia apparatus and to a respiratory flow sensor with a tubular housing and with a measuring insert that can be inserted into same with at least one hot wire.

### BACKGROUND OF THE INVENTION

Such respiratory flow sensors and especially hot wire anemometers are usually used in respirators and anesthesia apparatus in order to measure the gas volume flows from and to the patient being respirated and to draw conclusions on the patient's current status from the results of the measurements or to control the respiration and further treatment of the patient as a function of the results of the measurements.

A respiratory flow sensor of this type is described, e.g., in EP 0 024 327 B1.

Depending on the special design of the measuring insert with one or more hot wires and a suitable measured signal evaluation, the gas volume flows from and to the patient that are detected in the respiratory flow sensor can be determined in terms of amount and direction.

The hot wire anemometers used hitherto in medical engineering are either disposable sensors for a single-time use or are replaced after only a few applications for measurements and must be disposed of in each of the two cases mentioned.

In the prior-art hot wire anemometers, the measuring insert with the wires is rigidly connected to the tubular housing by means of a suitable adhesive. Attempts to autoclave the prior-art hot wire anemometers have failed because, due to the material stresses and expansions caused by the heat, the flow sensors are no longer gas-tight or their measuring properties are impaired. The consumption of such respiratory flow sensors has hitherto been correspondingly high, associated with correspondingly high costs for the purchase of a new unit and ultimately the environmental pollution involved in disposal.

If the prior-art respiratory flow sensors are used for a longer period of time, the measuring technical properties change and possibly lead to incorrect measurement results.

### SUMMARY AND OBJECTS OF THE INVENTION

The primary object of the present invention is to provide an improved, namely, reusable and autoclavable respiratory flow sensor with long durability of the type mentioned in the introduction.

According to the invention, a respiratory flow sensor with a tubular housing and with a measuring insert that can be inserted into the tubular housing along with at least one hot wire. The measuring insert is fastened replaceably in the housing by means of a snap connection and consists of a high-temperature plastic.

The snap connection may be formed by the cooperation of a projection of an opening in the housing with incisions in the measuring insert and with a sealing ring during the insertion of the housing. The measuring insert and/or the housing may be manufactured from one of the materials polysulfone (PSU), polyphenyl sulfone (PPSU) or polyamide. The measuring insert may have the hot wires mounted by means of a pair of the holding pins each, wherein the hot wires are arranged at different distances from the central longitudinal axis of the housing. One of the hot wires may be arranged in front of or behind an additionally arranged air resistance body when viewed in the direction of flow, so that the cooling effect of the respiratory flow flowing past on the hot wire is different in a direction-dependent manner and is thus used to recognize the direction of the respiratory flow.

The holding pins may be connected to the evaluating electronic unit by means of the contacts.

According to another aspect of the invention, a method is provided in which the respiratory flow sensor may be used in a respirator or anesthesia apparatus. The method may further include sterilizing the sensor, i.e., autoclaving with hot steam under pressure.

According to another aspect of the invention, a respiratory flow sensor is provided with a tubular housing and with a measuring insert that can be inserted into same with at least one hot wire. The measuring insert is replaceably fastened in the housing by means of a snap connection and consists of one of the materials polysulfone (PSU), polyphenyl sulfone (PPSU) or polyamide.

In the respiratory flow sensor according to the present invention, the measuring insert is connected to the tubular housing of the sensor detachably, i.e., replaceably by means of a mechanical snap connection. The tubular housing is designed especially in the form of a venturi tube, so that the gas inlet and gas outlet cross sections expand from the center of the sensor toward both ends of the sensor, and a section of minimal and constant cross section of the housing is present in the center of the sensor in the area of the measuring insert in order to obtain a good and reproducible measured signal resolution. Both the measuring insert and the housing are preferably manufactured from a high-temperature plastic that can be subjected to thermal and mechanical loads, especially from a material from the group comprising polysulfone (PSU), especially polyphenyl sulfone (PPSU), or polyamide. One advantage of the present invention is that the respiratory flow sensor according to the present invention can also be sterilized, i.e., autoclaved as a result with hot steam under pressure and it can be reused as a result. If the measuring insert is possibly defective after multiple use, it can be replaced and the tubular housing can be used further. Due to the reusability of the respiratory flow sensor, costs can be saved and environmental pollution can be diminished. There will be no deviations from the measuring accuracy even in case of long-term use because of the dimensional stability of the sensor design.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which a preferred embodiment of the invention is illustrated.

### BRIEF DESCRIPTION OF THE DRAWINGS

In the drawings:

FIG. 1 is a front view of a respiratory flow sensor according to the present invention in the direction of flow of the gas with the housing and the measuring insert; and

FIG. 2 is a longitudinal sectional view through the respiratory flow sensor according to the present invention corresponding to the section line A-A in FIG. 1.

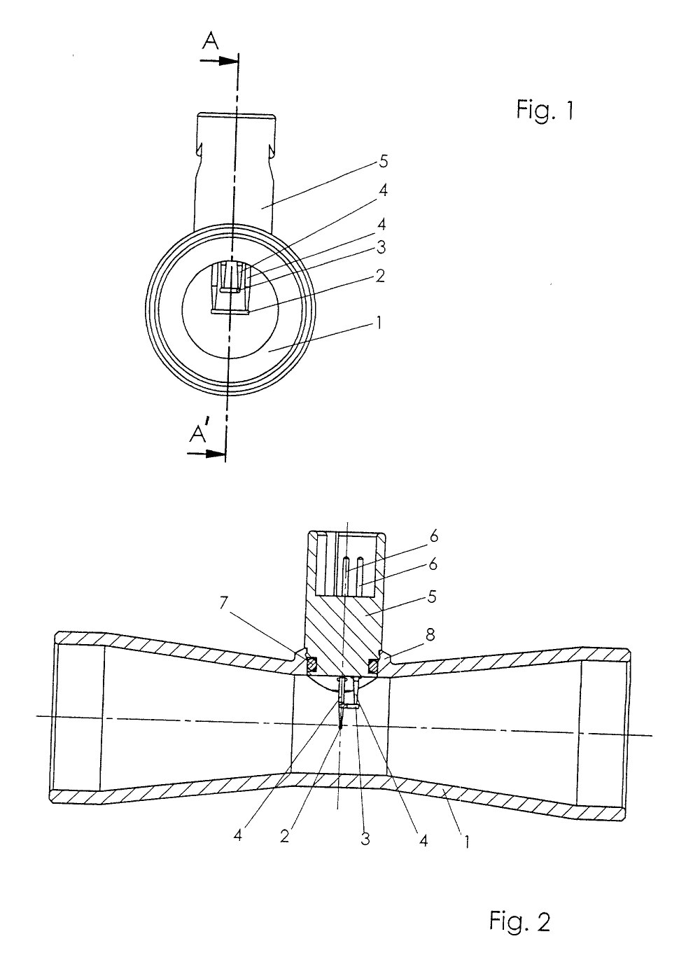

### DESCRIPTION OF THE PREFERRED EMBODIMENT

Referring to the drawings in particular, the breathing gas to be measured flows through the tubular housing 1. The tubular housing 1 is designed in the form of a venturi tube with a minimal and constant cross section in the area of the measuring insert 5. The breathing gas flowing through the housing 1 cools an electrically heated first hot wire 2 as a function of the gas volume flow flowing past, and the temperature-dependent change in the resistance of a second hot wire 3 is detected and, calculated with the measured signal of the first hot wire 2, it is used to compensate the temperature effect on the gas volume flow of the breathing gas which is finally determined. The signal voltage of the first hot wire 2, which is proportional to the breathing gas volume flow, is converted into the temperature-corrected breathing gas volume flow by means of a prior-art evaluating electronic unit and evaluating software. The hot wires 2, 3 are arranged on holding pins 4, which are part of the measuring insert 5, which has contacts 6 for the outer contacting. In a special embodiment with two heated hot wires of such a respiratory flow sensor, an air resistance body may also be additionally provided, so that the direction of the gas volume flow can be detected based on the difference in the intensity of the cooling effect on the hot wires 2, 3 exposed to the breathing gas flowing past to a greater or lesser extent.

The measuring insert 5 and preferably also the housing 1 are made of a high-temperature plastic that can be subjected to thermal and mechanical stress, the especially suitable materials being selected from the group of the polysulfones (PSU), especially polyphenyl sulfone (PPSU), or the polyamides of various hardnesses and filler contents. The measuring insert 5 is replaceably fastened in the housing 1 by means of a snap connection.

The snap connection is sealed with an elastic and preferably likewise heat-resistant sealing ring 7, so that all components of the respiratory flow sensor according to the present invention can be autoclaved. The sealing ring 7 is manufactured especially from a silicone or rubber material.

As is shown in FIG. 2, the snap connection is designed such that a defined, gas-tight, but detachable unit comprising the housing 1 and the measuring insert 5 is formed by the cooperation of the preferably circular projection 8 of a circular opening of the housing 1 with incisions of the measuring insert 5 and with the elastic sealing ring 7.

In the exemplary embodiment, the housing 1, designed as a venturi tube, has a length of about 76 mm, a maximum internal diameter of about 19 mm and a minimum internal diameter of about 13 mm.

While specific embodiments of the invention have been shown and described in detail to illustrate the application of the principles of the invention, it will be understood that the invention may be embodied otherwise without departing from such principles.

What is claimed is:

1. A respiratory flow sensor, comprising:
a tubular housing; and
a measuring insert formed of a high-temperature plastic that can be inserted into said tubular housing, said measuring insert having at least one hot wire, said measuring insert being fastened replaceably in said tubular housing via a snap connection.

2. A respiratory flow sensor in accordance with claim 1, wherein the snap connection is formed by the cooperation of a projection of an opening in the housing with incisions in the measuring insert and with a sealing ring during the insertion of the housing.

3. A respiratory flow sensor in accordance with claim 1, wherein the measuring insert and/or the tubular housing are manufactured from one of the materials polysulfone (PSU), polyphenyl sulfone (PPSU) or polyamide.

4. A respiratory flow sensor in accordance with at least claim 1, wherein the measuring insert has the hot wires, the hot wires being mounted by means of a pair of holding pins each, wherein the hot wires are arranged at different distances from the central longitudinal axis of the housing.

5. A respiratory flow sensor in accordance with claim 4, further comprising: air resistance body wherein one of the hot wires is arranged in front of or behind the air resistance body when viewed in the direction of flow, so that the cooling effect of the respiratory flow flowing past on the hot wire is different in a direction-dependent manner and is thus used to recognize the direction of the respiratory flow.

6. A respiratory flow sensor in accordance with claim 4, wherein the holding pins are connected to the evaluating electronic unit by means of the contacts.

7. A respiratory flow sensor, comprising a tubular housing and a measuring insert that can be inserted into said tubular housing with at least one the hot wire wherein the measuring insert is replaceably fastened in the housing by means of a snap connection, the measuring insert being formed of one of the materials polysulfone (PSU), polyphenyl sulfone (PPSU) or polyamide.

8. A method of using a respiratory flow sensor, the method comprising the steps of:
providing a tubular housing; and
providing a measuring insert formed of a high-temperature plastic, said measuring insert having at least one hot wire;
inserting the measuring insert into said tubular housing;
fastening the measuring insert replaceably in the tubular housing via a snap connection; and
connecting the respiratory flow sensor to a respirator or anesthesia apparatus.

9. The method in accordance with claim 8, wherein the snap connection is formed by the cooperation of a projection of an opening in the housing with incisions in the measuring insert and with a sealing ring during the insertion of the housing.

10. The method in accordance with claim 8, wherein the measuring insert and/or the tubular housing are manufactured from one of the materials polysulfone (PSU), polyphenyl sulfone (PPSU) or polyamide.

11. The method in accordance with claim 8, wherein the measuring insert has the hot wires, the hot wires being mounted by means of a pair of holding pins each, wherein the hot wires are arranged at different distances from the central longitudinal axis of the housing.

12. The method in accordance with claim 8, further comprising using an air resistance body with one of the hot wires is arranged in front of or behind the air resistance body when viewed in the direction of flow, so that the cooling effect of the respiratory flow flowing past on the hot wire is different in a direction-dependent manner and is thus used to recognize the direction of the respiratory flow.

13. The method in accordance with claim 11, wherein the holding pins are connected to the evaluating electronic unit by means of the contacts.
